(19) United States
(12) Patent Application Publication
(10) Pub. No.: US 2026/0232904 A1
(43) Pub. Date: Aug. 13, 2026

(54) Methods Assemblies Devices Systems and Functionally Associated Machine Executable Instructions for Assessing Responsiveness of a Subject to Infusion of Liquids

(71) Applicant: FIZE MEDICAL LTD., Modin (IL)

(72) Inventor: Noam Levine, Modin (IL)

(21) Appl. No.: 19/050,463

(22) Filed: Feb. 11, 2025

(51) Int. Cl. A61M 5/172

(52) U.S. Cl. CPC A61M 5/1723; A61M 2205/3379

(57) ABSTRACT

Disclosed are methods, assemblies, devices, systems and functionally associated machine executable code for assessing a subject's responsiveness to infusion of fluids. Embodiments of the present invention may include a medical device having a first interface to an electronically controllable infusion pump, a second interface to a urine output monitor, and control logic to: (a) cause said infusion pump to modulate an infusion pattern or signal onto fluid flow being infused into a subject; and (b) examine urine output measurement data from said urine output monitor for the effect of the infusion pattern on the urine output.

### FIELD OF THE INVENTION

The present invention relates generally to the field of medical devices and systems. More specifically, the present invention relates to methods, assemblies, devices, systems, and functionally associated machine executable instructions for assessing responsiveness of a subject to infusion of (therapeutic) liquids.

### BACKGROUND

Critically ill patients in the intensive care unit (ICU) routinely require intravenous (IV) fluid administration to maintain adequate tissue perfusion and organ function. However, determining and titrating the optimal fluid amount to keep adequate fluid balance is a persistent challenge. Both inadequate fluid replacement (hypovolemia) and fluid overload can have deleterious effects.

Hypovolemia starves organs and tissues of oxygen and nutrients, potentially worsening organ dysfunction, while excessive fluids accumulate in the tissues (edema) and lungs, impeding gas exchange and oxygenation. Therefore, accurate assessment of a patient's volume status and ongoing fluid needs is crucial.

Several clinical parameters like heart rate, blood pressure, and urine output are conventionally monitored. However, these can lack sensitivity and specificity for detecting early or marginal intravascular volume deficits or excesses. The current gold standard is to perform functional hemodynamic tests that assess the cardiovascular system's responsiveness to fluid boluses.

One such technique is the fluid challenge, which involves rapid IV infusion of about 500 mL crystalloid solution over 10-30 minutes, while monitoring dynamic changes in cardiac output like stroke volume variation (svv), oxygen delivery, or surrogate measures like arterial pressure waveforms. An increase in these parameters indicates the patient is fluid responsive and will benefit from more fluids, while lack of improvement implies further fluids may be detrimental.

An alternative test is the furosemide stress test utilizing IV furosemide, a commonly used loop diuretic. Furosemide inhibits sodium and chloride reabsorption in the ascending limb of the Loop of Henle in the nephrons, leading to natriuresis and diuresis. A brisk urine output following furosemide in a previously oliguric patient confirms preserved renal perfusion and rules out hypovolemia as the etiology.

However, the main dilemma is the risks associated with choosing the wrong test. Erroneously performing fluid challenge in fluid overloaded states like heart failure or renal failure can precipitate pulmonary edema. Meanwhile, incorrectly selecting furosemide stress testing in hypovolemic patients can lead to critical reductions in intravascular volume. This conundrum continues to plague even experienced intensivists on a regular basis. More accurate, real-time and less harmful predictors of volume responsiveness are needed.

### SUMMARY OF THE INVENTION

Embodiments of the present invention may include methods, assemblies, devices, systems, and functionally associated machine executable instructions for assessing responsiveness of a subject to infusion of fluids including therapeutic substances in liquid forms. According to an embodiment of the present invention, a subject's kidney urine production or urine output rate may be measured, tracked and correlated to an infusion pattern detected or imposed on fluids being infused into the subject. Systems according to embodiments for assessing fluid responsiveness may measure and or detect an infusion pattern inherently produced by one or more pumps connected to the subject. Additionally, such systems under certain circumstances, such as for example when no clear inherent infusion pattern is detectable during normal infusion pump operation, may signal, induce or otherwise cause one or more infusion pumps to modulate a pattern of infused fluid of their respective outputs.

Systems according to embodiments of the present invention may include: (1) one or more blader output (urine) sensors with electrical signal output, (2) one or more infusion pumps with a digital interface to provide remote access and control of the infusion pumps control systems, (3) one or more dedicated controllers or multi-purpose processors integral or otherwise functionally associated with a first external interface to facilitate communication between the controller and the one or more sensors, and a second external interface to facilitate communication between the controller and the one or more infusion pump. A controller or multi-purpose processor according to embodiments of the present invention may, among many other functions, monitor output signal from the one or more bladder/urine output sensors and may both monitor and regulate operating states, modes and output patterns of each of one or more infusion pumps.

System controllers according to further embodiments of the present invention may receive, aggregate, analyze and use information provided by the one or more sensors and one or more infusion pumps. The aggregated information correlating infusion types and rates with measured urine output may be either passively measured or detected, or actively reviled through infusion modulation. The aggregated information may be analyzed in order to: (1) estimate various parameters relating to a specific patient or subject of an infusion session; (2) generate notifications relating to estimated patient/subject parameters, and/or (3) initiate one or more diagnostic processes on the patient/subject using the infusion pump and urine sensor combined information.

As part of a diagnostic process according to embodiments of the present invention, a system controller may estimate fluid responsiveness of a subject receiving one or more infusions. More specifically, over a defined period, the system controller may estimate or otherwise determine a patient's response to individual and or aggregated infusion flowrates and/or patterns from one or more infusion pumps. According to embodiments, the system controller may monitor and track a subject's urine output over time.

Despite propagation delays between infused fluids and the urine the fluid induces, correlation of measured output patterns to specific infusion volumes and/or patters may be achieved using a micro-challenge technique. The micro-challenge technique may include modulation of infusion rates from a specific infusion pump or combination of pumps in a pattern such that the modulation induces little to no change in aggregated or averaged fluid infusion experienced by a patient over a clinically meaningful period of time, such as several minutes or several hours.

A comparison of propagation delays and waveform transformations experienced by a micro-challenge infusion volume (i.e. a modulated infusion fluid signal) as measured by the one or more urine output monitors relative to an original infusion signal induced through the one or more infusion pumps facilitates an estimation of a subject's hydration condition and or fluid responsiveness. According to further embodiments, the controller may also compare the composition of infused fluid(s) in each of one or more infusion pumps, against the rate of propagation and waveform transform of a micro-challenge pattern infused into a subject. When urine output is measured by sensors on the urine collection side following a micro-challenge type infusion, embodiments of the present invention can extrapolate micro-challenge propagation variances and waveform transformation as a function of infusion, which variances and transforms may provide insights into the presence or absence of various health related parameters and or conditions afflicting the subject.

System controllers according to embodiment of the present invention may implement or otherwise utilize various protocols to estimate a subject's fluid responsiveness from operational parameters and outputs of functionally associated infusion pumps and or fluid flow sensors. According to some embodiments, system controllers may determine or estimate one or more of a subject's fluid responsiveness with only the output of the urine flow sensors, detecting and interpreting an infusion pattern automatically or inherently modulated onto the infusion stream due to the nature of the pump and or pumping regime for a specific therapeutic being infused.

According to further embodiments, operating system controllers may determine infusion pump rates and patterns through direct communication with infusion pumps when fluid infusion patterns or signals from urine flow sensors are unclear or unreliable. This communication may be established with one or more infusion pumps during an active session. The system controller can both send commands to adjust pumping rates and receive pumping rate data from the pumps. In some embodiments, this communication channel may be bidirectional, enabling both control and monitoring functions through a single interface.

According to further embodiments, induced modulation of fluid infusion, i.e. changing rate of fluid infusion based on a known pattern, may be continuous, intermittent, timed, and/or triggered by an operator or by some detection of some events or conditions relating to the subject. According to further embodiments, the infused pattern may not be known in advance but may at least partially be a function of one or more measured urine output flow parameters. Detection of one or more urine flow parameters correlated with a specific modulated or inherently produced, fluid infusion pattern within time series measurements of kidney output of a subject whose fluid infusion was modulated, or infusion pattern was inherently produced using the same or another specific pattern may indicate the responsiveness of a subject to fluid infusion. Although the modulated or inherently produced infusion pattern will not necessarily be reflected 1:1 in the detected urine production, different modulations and/or different inherently produced infusion patterns of different fluids and or medications and different patient responsiveness may affect measured urine output parameters, including: 1. the time until the urine rate responsively changes; 2. the slope of the change; 3. the amplitude of the change; 4. the curvature, or second derivative, of the change; and 5. any combination of 1 through 4.

Alternatively, failure to detect any urine flow related parameters changes corresponding to detected or modulated infusion patterns of certain fluids may suggest the subject is marginally or nonresponsive to fluid infusion or to certain fluid(s) infusion or may be suffering from some other fluid imbalance condition. For example, an overloaded patient will generally not respond to fluids, so the patient is not fluid responsive, but will respond to diuretics. One of the benefits resulting from the induced modulation embodiments of the present invention is that the controller can modulate different types of fluids/medications, individually or in parallel, and the controller can assess a subject's different response to each of the pump/fluid/medication modulations, individually or in combination.

Medical devices and systems in accordance with embodiments of the present invention may include a urine output rate measurement device and an injection pump controller, integral or otherwise functionally associated with the measurement device. The injection pump controller may be communicatively coupled with and instruct or otherwise regulate operation of one or more infusion pumps associated with, i.e. infusing, a subject/patient whose urine output is monitored by the measurement device. Such a closed loop system may select a first infusion rate or pattern at least partially based on a measured urine production rate or pattern and or some mathematical understanding, rules or model indicating what injection rate or pattern should correspond to the measured urine rate or pattern. Second and subsequent infusion rates and or patterns may be selected depending on the subject's response to the first injection rate.

According to further embodiments, the injection pump controller, when selecting a fluid injection rate or modulation scheme (e.g. change in rate over time like a fluid injection signal or pattern over time) for a subject, may factor a variety of other parameters including a doctor's fluid(s) administration instructions, values of various measured vital signs, and other medically significant indicators recorded within a subject's electronic medical records. The injection rate or modulation scheme/signal/pattern may be altered by logic integral or otherwise functionally associated with the injection pump controller for a subject or patient in response to how the patient responds to the selected rate or modulation scheme/signal/pattern.

Embodiments of the present invention may include methods, assemblies, devices, systems and functionally associated machine executable instructions for continuously estimating fluid responsiveness in critically ill patients receiving intravenous (IV) fluids/medications. A system, closed-loop or open-loop may attempt to correlate modulating patterns of the patient's fluid(s) infusion rates to one or more parameters changes of a monitored urine output to assess fluid responsiveness/status of the patient. According to closed-loop implementations, the injection pump controller may dynamically adjust and adapt the infusion rate modulation pattern employed based on real-time analysis of the subject's urine output response, thereby continually optimizing the induced pattern to more accurately determine fluid responsiveness/status. In some further embodiments, the injection pump controller may dynamically adjust and adapt the infusion rate modulation pattern employed based on real-time analysis of the subject's urine output response and one or more other parameters (e.g. blood pressure).

A system according to embodiments of the present invention may include a urine output monitoring device, such as a bladder catheter connected to a urine meter, which provides ongoing urine flow rate data. An infusion pump controller may communicate with infusion pumps connected to the patient in order to check and optionally to modulate the pumps' infusion rates, individually and collectively. According to further embodiments, subtle fluctuations may be induced in the total infusion rate by changing one or more individual pump rates while staying within the physician's prescribed amounts.

The infusion rate modulation according to embodiments of the present invention may follow predetermined patterns of periodic fluctuations or “signals” in either individual pump rates and or in total infusion rates across a set of pumps. The patterns may involve gradual or rapid increases and decreases with specific shapes, amplitudes, frequencies, and durations. Unlike prior art fluid challenge tests that only increase fluids, embodiments of the present invention may inspect and analyze urine output responses to both increasing (positive) and decreasing (negative) rates of the infusion patterns across the pumps, which may facilitate fluid responsiveness testing of a patient during reduction of fluids as well as during fluid administration increases.

More specifically, embodiments of the present invention may induce various modulation patterns in the fluid infusion process, tailored to elicit measurable responses in urine output that correlate directly to the subject's infused therapeutic fluid responsiveness. These modulations are not arbitrary but may instead be strategically designed to cover a broad spectrum of physiological responses. Patterns of modulation may include periodic modulations (e.g., sinusoidal, square, and triangular waveforms), step changes, ramp modulations, pulse modulations, random or stochastic modulations, combinations thereof, and patient-specific modulations. This diversified approach to infusion modulation may provide for a comprehensive assessment of fluid responsiveness, accommodating for the unique physiological dynamics of as many subjects as possible.

Once an infusion modulation pattern for a patient is established, either through monitoring or through induced pattern modulation, or through combination thereof, output data from a functionally associated urine meter connected to the same patient or subject may be collected, inspected and analyzed, in real-time or with a delay, to detect changes in one or more urine flow rate parameters which may “match” or be otherwise correlated with infusion flow changes corresponding to the established, detected or induced, infusion patterns for that patient.

Detection of one or more urine flow parameters correlated with a specific modulated fluid infusion pattern may indicate the subject's fluid status is relatively responsive and/or the responsiveness level. These parameters can include the time (propagation) delay until urine rate changes, the slope of the urine rate change, the amplitude/magnitude of the change, the curvature or higher derivatives of the change profile, as well as potential differences in these parameters for positive versus negative infusion rate changes. These parameter changes may be highly specific to the kinds of medication being pumped. For example, a non-fluid responsive patient can have strong urine flow correlation to Furosemide (diuretic) modulation, or to other medication modulation.

Conversely, a low correlation between infusion modulation and urinary output parameters may indicate low fluid responsiveness to specific fluids or medications.

According to embodiments of the present invention, the urine flow monitor and the controller logic can jointly measure and track correlation of kidney output flow rate parameters to modulation in the at least one fluid infusion pattern(s). The tracked urine flow parameters may include one or more of the following key urine flow characteristics:
(a) Time to Response: The delay between the initiation of an infusion modulation or a signal and the observable change in urine output. This parameter can help in understanding the responsiveness (e.g. speed) of the renal system and other organs to changes in fluid administration;
(b) Rate of Urine Output: The absolute change in urine production rate following an infusion pattern modulation. An increase or decrease in urine output can directly reflect the subject's fluid balance and responsiveness;
(c) Volume of Urine Output: The total volume of urine produced over a specific time frame after modulation. This can provide insights into the overall fluid status and efficiency of the renal system and other organs related to processing and excreting fluids;
(d) Slope of Urine Rate Change: The rate at which urine output changes over time, which can be indicative of the kidney's ability to adapt to changes in fluid volume and pressure;
(e) Amplitude of Urine Rate Change: The magnitude of change in urine output rate, which reflects the renal system's capacity to respond to variations in fluid administration;
(f) Curvature or Second Derivative of the Urine Rate Change: This measures the acceleration or deceleration of the change in urine output rate, offering insights into the dynamic responses of the renal system to fluid infusion modulations;
(g) Differences in Response to Positive versus Negative Infusion Rate Changes: This involves comparing the renal response to increases (positive changes) and decreases (negative changes) in fluid infusion rates, which can reveal the system's sensitivity and adaptability to different types of fluid management strategies;
(h) Urine Specific Gravity or Osmolality Changes: Changes in the concentration of urine, which can indicate the kidney's ability to concentrate or dilute urine in response to fluid administration, thereby reflecting fluid and electrolyte balance;
(i) Variability in Urine Output: The degree of fluctuation in urine output over time, which might indicate the stability or instability of the patient's fluid status and the effectiveness of the modulation strategy.

These parameters, when analyzed individually and especially in concert, can provide a comprehensive picture of a patient's fluid status, enabling tailored and dynamic fluid management strategies. By closely monitoring these urine flow parameters in response to specific infusion flow modulations, healthcare providers can optimize fluid therapy, enhance patient outcomes, and minimize the risk of complications associated with fluid imbalance. These parameters, when analyzed individually and especially in concert, can additionally or alternatively provide a comprehensive snapshot of the patient's various organs and their function level. By closely monitoring these urine flow parameters in response to specific infusion flow modulations, healthcare providers may be able to detect and/or predict specific organ failure or dysfunction.

A system according to embodiments of the present invention may include machine learning algorithms that are trained to recognize specific signatures composed of combinations of urine output patterns and modulations associated with different types and degrees of a subject's fluid status. Additional patient vital sign data and lab results may also be incorporated into the algorithms which factors some or all the provided medical information to derive, extrapolate or inference a condition of one or more organs of the patient. The controller's infusion modulation pattern can be adapted based on algorithmic analysis and feedback to optimally assess and track changes in fluid balance. Different modulation patterns may be used in order to gather data usable for checking the conditions of different organs and or biological processes of the patient.

Embodiments of the present invention may help mitigate the risks of traditional larger-volume intermittent fluid challenge tests and testing systems. It may provide continuous quantitative fluid status evaluation to optimize early goal directed therapy and potentially reduce morbidity and mortality. It may function effectively even with standard polyuric ICU patients. The responsiveness specificity for predicting fluid or diuretic effects may exceed existing approaches.

According to various embodiments of the invention, it may be important to optimize the administration of fluids and medications in a manner that avoids counterproductive interactions and enhances the diagnostic effects of the fluids and medications. For example, the concurrent administration of diuretics, which promote urine production, alongside medications that conserve body fluids, may be contraindicated as they exert opposing physiological effects. Thus, the system controller according to embodiments may include protocols to avoid conflicting infusions and to strategically coordinate the infusion of fluids and diverse medications such as vasopressors and inotropic agents, ensuring that their interactions amplify beneficial outcomes, such as urine response, rather than counteract each other.

In certain embodiments, the system controller may employ a tailored approach to micro fluid or medication challenges based on the real-time assessment of a patient's fluid status. For instance, if the system determines that the patient is positioned higher on the Frank-Starling curve, suggesting a decreased responsiveness to fluid administration, micro-challenges involving diuretics like furosemide may be initiated. This approach could yield more pronounced and observable changes in urine output, thereby providing clearer insights into the patient's fluid responsiveness.

Furthermore, embodiments of the present invention may involve sequencing micro fluid or medication challenges that have opposite effects on urine production in a consecutive order. By administering a diuretic challenge followed immediately by an antidiuretic challenge, and vice versa, the system can significantly enhance the detectability of changes in urine production, thereby facilitating a more precise assessment of the patient's fluid balance status.

Additionally, according to some embodiments, a system controller may adjust the infusion rates of different medications in an alternating pattern. By increasing the rate of one medication while decreasing the rate of another, and subsequently reversing these adjustments, the system may not only achieve a minimal net change in overall infusion rate but also enhance the effects on urine production. This dual modulation approach may allow for a continuous and dynamic assessment of fluid responsiveness, providing insights that enable timely and effective adjustments to the treatment regimen.

These strategies, implemented according to embodiments of the present invention, may enable a dynamic and responsive approach to managing fluid and medication therapies. By ensuring that each intervention is both timely and effectively aligned with the patient's evolving therapeutic needs, the system may optimize patient outcomes and minimize the risks associated with fluid imbalance and medication interactions.

According to further embodiments, employed machine learning algorithms may be trained on a database of urine output signatures associated with different drug types, medications, and fluid compositions. These algorithms may also factor in a variety of other vital signs and medical parameters relating to a patient. This training may possibly be used with a system according to embodiments to account and compensate for varying effects different infused substances can induce on kidney function and various other organs such as the heart which affect urine production, when analyzing fluid responsiveness and/or adjusting infusion levels.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a functional block diagram illustration of a fluid responsiveness assessment system according to embodiments of the present invention;

FIG. 2 is a functional block diagram of an exemplary device controller in accordance with embodiments of the present invention;

FIGS. 3A and 3B is a set of diagrams depicting the flow of infused fluid from a set of infusion pumps, individually and collectively;

FIG. 3C is a diagram depicting the collective flow of infused fluid from all the pumps with the addition of an induced modulation pattern in accordance with embodiments of the present invention;

FIG. 4 is an exemplary diagram showing a patient's fluid status on the Frank-Starling curve; and

FIG. 5 is a flow diagram defining an exemplary set of steps performed by a controller in accordance with embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any computer type or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any programming language. It will be appreciated that a variety of programming languages may be used to implement the teachings of the inventions as described herein.

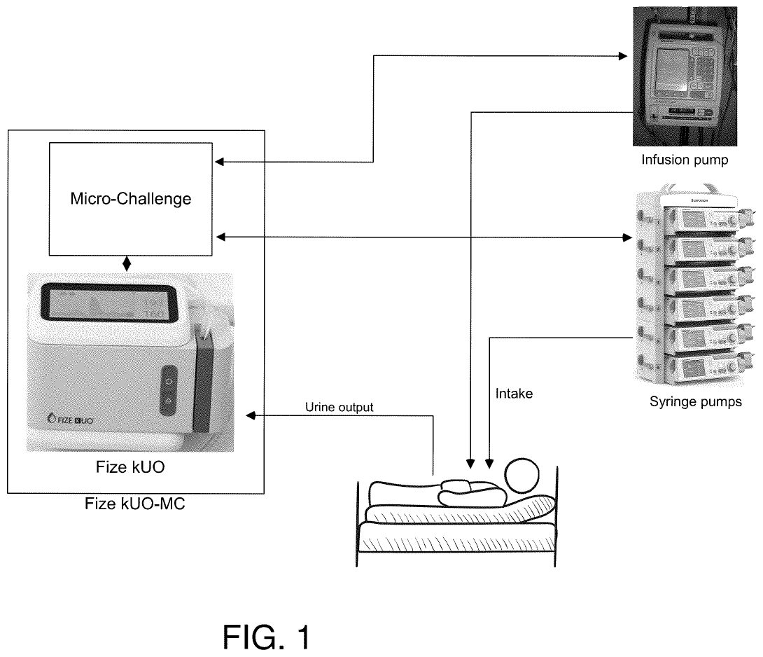

Turning now to FIG. 1, there is shown a functional block diagram illustration of a fluid responsiveness assessment system according to embodiments of the present invention. At the core of the system is a controller, designated as a “Micro-Challenge”, communicatively coupled with a set of pumps, infusion and syringe, and with a kidney output monitor, designated as make& model: Fize kUO. The Micro-Challenge controller coupled with a Fize kUO is designated as Fize kUO-MC. The microcontroller is configured to receive, transmit, or both receive and transmit message with each of the pumps and with the kidney output flow monitor. By reading the messages from the one or more pumps, the controller is able to track fluid infusion into a subject, search the flow for detectable infusion patterns which might translate into detectable urine outflow patterns or urine flow parameters. Measured urine flow messages received by the controller from the Fize kUO may be tracked to detect for any infusion pattern or infusion pattern related parameters to gauge a subject's fluid responsiveness. The controller may also or alternatively control the pumps to induce infusion patterns.

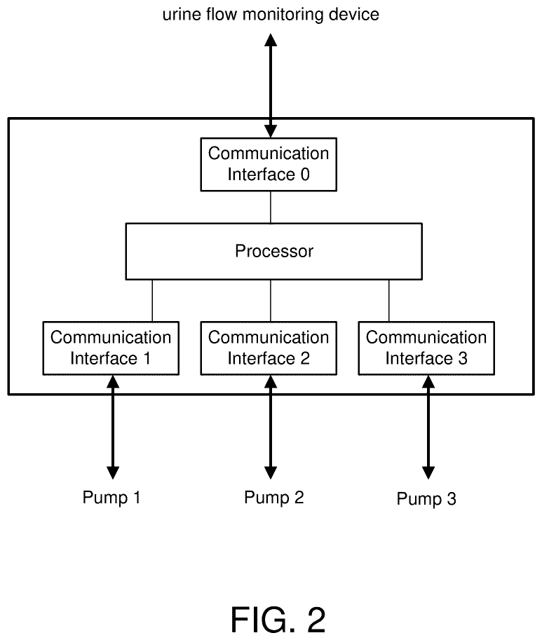

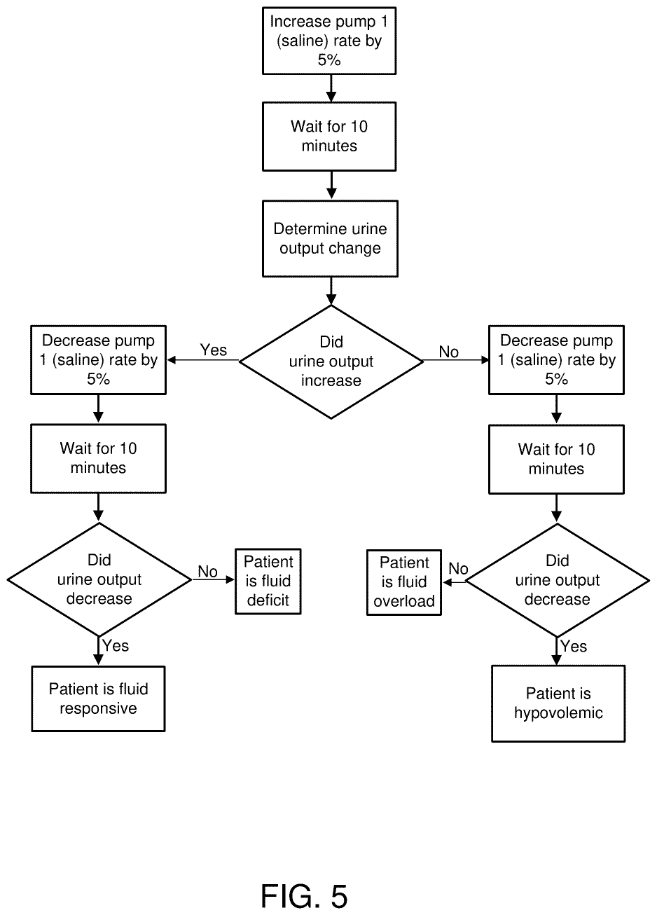

Turning now to FIG. 2, there is shown a functional block diagram of an exemplary device controller in accordance with embodiments of the present invention. The controller's structure and operation may best be described in conjunction with the steps listed in the flowchart of FIG. 5. The controller includes: (a) communication interfaces to one or more pumps and to a urine flow monitoring device; (b) The controller may include custom logic circuits or a programmed general purpose processing circuit to perform each of one or more fluid responsiveness tests of a subject using the connected and controllable pumps along with feedback from the kidney output monitor. The flow chart of FIG. 5 illustrates an exemplary step flow of steps to be executed by the controller to assess subject/patient responsiveness.

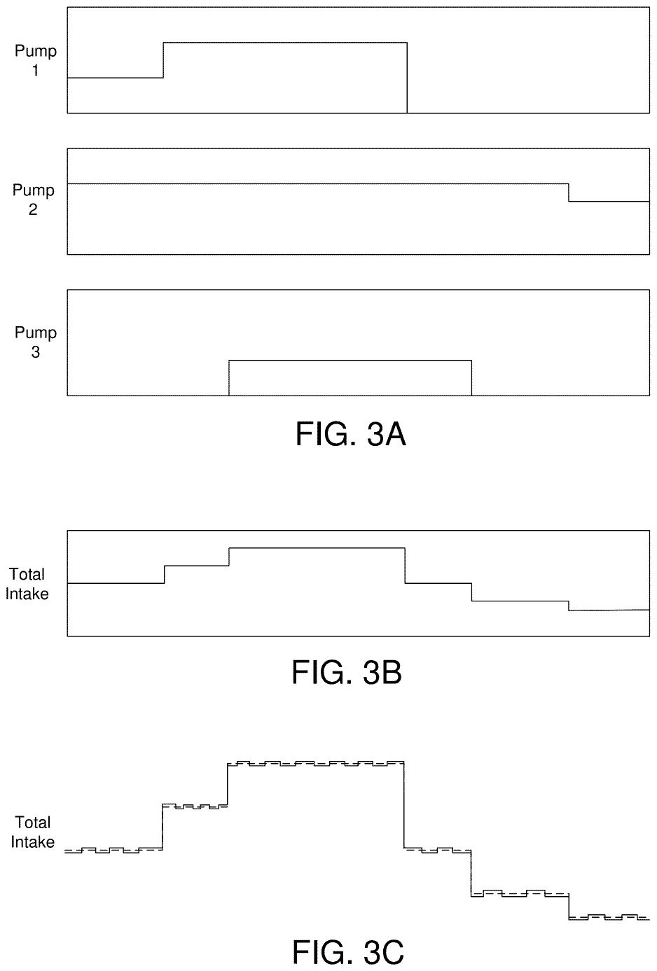

The exemplary flow of steps illustrated within FIG. 5 is associated with a process termed “micro-challenge” to be performed by a controller like the one shown in FIG. 2. As is evident from FIGS. 2 and 3A to 3D, modulation of fluid within a micro-challenge process involves inducing known/predefined changes to infusion pump outputs and concurrently monitoring for resulting changes in urinary output amongst other vital signs. The urinary pattern corresponding to the modulated infusion is usually delayed and somewhat transformed from the modulated infusion pattern. There is information about the patient to be derived from both the delay time and the nature of transformation induced on the infusion pattern of the micro-challenge.

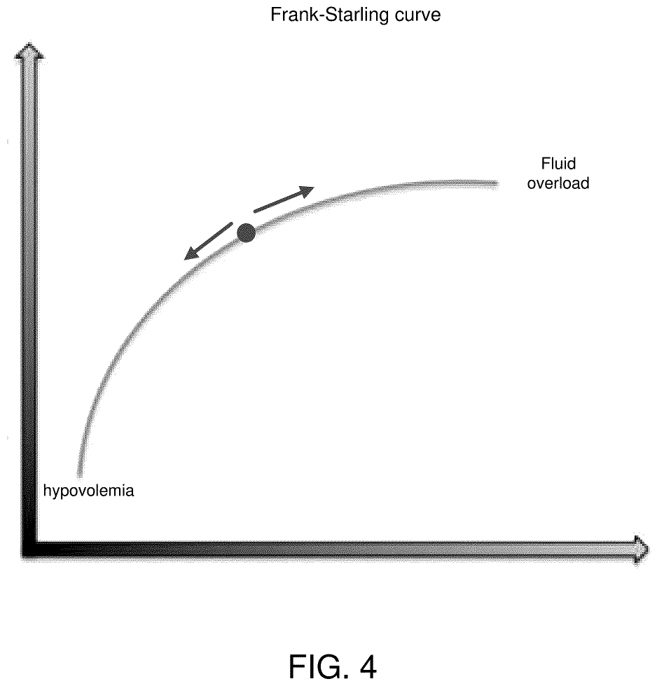

Following the steps of FIG. 5 or by implementing other embodiments described in this invention, it is possible to communicate to the doctor a patient's hydration condition by using the Frank-Starling curve graph of FIG. 4. By projecting, possibly displaying, the patient's fluid status as a dot on the Frank-Starling curve, the dot's location indicates the patient's fluid status. The more the patient is fluid overloaded, the more up and to the right the dot moves on the curve. While conversely the more the patient is dehydrated, the more down and to the left the dot is located on the graph.

Turning now FIG. 4, there is shown a Frank-Starling graph representing the relationship between preload and cardiac output, wherein a movable indicator dot is positioned on said curve to represent a patient's current fluid status. In the context of systems according to the embodiments of the present invention, the position of the indicator dot is updated based on the patient's measured responses to coordinated micro-challenges with fluids and medications, wherein rightward movement along the curve's x-axis indicates increasing fluid overload and leftward movement indicates increasing hypovolemia.

In some embodiments, the system utilizes the non-linear properties of the Frank-Starling relationship to optimize timing and selection of fluid and medication micro-challenges, wherein the curve's steeper left portion indicates heightened responsiveness to fluid administration in hypovolemic states, and the flatter right portion indicates reduced responsiveness in fluid overload states. The differential responsiveness across curve regions enables the system to adaptively select appropriate interventions, for example, preferentially administering diuretic challenges when the indicator shows positioning on the curve's right portion, or fluid challenges when positioned on the left portion.

Systems according to further embodiments of the present invention leverage the curve's properties to orchestrate temporally opposed medication pairs, wherein agents with opposing effects on fluid status are administered sequentially to maximize observable changes in output measurements. For example, administration of a diuretic followed by an antidiuretic hormone produces enhanced measurement signals by exploiting the additive nature of their opposing effects on urine output, while maintaining zero net change in overall infusion rates through coordinated adjustment of multiple medication delivery rates.

According to some embodiments, the controller may track and identify that the pumps are infusing fluids according to patterns as shown in FIG. 3A. The controller may determine that the collective pattern of the three pumping patterns is the pattern shown in FIG. 3B. If the controller determines that the default infusion pattern created will not result in urine flow parameters sufficiently recognizable by measurement, the controller may choose to induce an additional modulation as is shown by the solid lines in FIG. 3C. The dashed line in FIG. 3C is the collective pattern shown in FIG. 3B.

The controller modulation logic may have the option to use one or more of a variety of infusion fluid pattern modulations, optionally found to optimize the assessment of a specific subject's fluid responsiveness. The illustrated system employs electrically controllable infusion pumps indirectly, through the controller, interfaced with a kidney urine output monitor, wherein the control logic is programmed to induce specific types of modulation in infusion patterns produced by the infusion pumps. These modulations may be of a type including, but are not limited to:
Periodic Modulations: Achieved by varying the infusion rate according to predefined waveforms, such as sinusoidal, which are selected for their frequency and amplitude characteristics. This pattern is useful for assessing the subject's response to predictable variations in fluid intake.
Step Changes: Characterized by abrupt alterations in the infusion rate, providing insight into the subject's immediate response to sudden increases or decreases in fluid delivery.
Ramp Modulations: Involves a gradual adjustment of the infusion rate, either increasing or decreasing over a specified period, to observe the effects of slowly changing fluid volumes.
Modulations: Consists of brief, intense fluctuations in infusion rate, interspaced with periods of standard infusion, useful for evaluating transient urinary system responses.
Random or Stochastic Modulations: Utilizes unpredictable variations in the infusion rate to simulate real-world fluctuations and assess the system's response to non-periodic changes in fluid administration.
Patient-Specific Modulations: Tailored modulation patterns based on the individual's specific physiological responses and historical data, ensuring personalized fluid management.
Adaptive modulations: Modulation patterns that are parameterized according to the kidney urine output, or to the kidney urine output response to the preceding modulation.
Combined modulation: Modulation pattern of multiple medication infusions. This modulation takes into account the different urine production response to different medications.

This diverse set of possible infusion pattern modulations facilitates a nuanced understanding of fluid responsiveness, allowing for precise adjustments to fluid therapy that are directly informed by real-time data and predictive analyses. Through this innovative approach, the invention illustrated in FIG. 1 offers a significant advancement in the field of critical care, enhancing patient outcomes through optimized fluid management.

The controller may also monitor the urine flow output for one or more of the following parameters:
Time to Response: This parameter measures the delay between the initiation of an infusion modulation and the observable change in urine output. It helps understand the responsiveness speed of the renal system to changes in fluid administration.
Rate of Urine Output: This reflects the absolute change in urine production rate following an infusion pattern modulation. An increase or decrease in this rate directly reflects the subject's fluid balance and responsiveness.
Volume of Urine Output: This parameter measures the total volume of urine produced over a specific timeframe after modulation. It provides insights into the overall fluid status and efficiency of the renal system in processing and excreting fluids.
Slope of Urine Rate Change: This indicates the rate at which urine output changes over time, indicative of the kidney's ability to adapt to changes in fluid volume and pressure.
Amplitude of Urine Rate Change: This measures the magnitude of change in urine output rate, reflecting the renal system's capacity to respond to variations in fluid administration.
Curvature or Second Derivative of the Urine Rate Change: This parameter measures the acceleration or deceleration of the change in urine output rate, offering insights into the dynamic responses of the renal system to fluid infusion modulations.
Differences in Response to Positive versus Negative Infusion Rate Changes: This involves comparing the renal response to increases (positive changes) and decreases (negative changes) in fluid infusion rates, revealing the system's sensitivity and adaptability to different types of fluid management strategies.
Urine Specific Gravity or Osmolality Changes: Changes in the concentration of urine can indicate the kidney's ability to concentrate or dilute urine in response to fluid or other medication administration, thereby reflecting fluid and electrolyte balance.
Variability in Urine Output: This parameter measures the degree of fluctuation in urine output over time, which might indicate the stability or instability of the patient's fluid status and the effectiveness of the modulation strategy.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

What is claimed:

1. A medical device comprising:
a first interface to one or more electrically controllable infusion pumps;
a second interface to a kidney urine output monitor; and
control logic to: (a) detect or induce an infusion pattern of fluid infused into a subject by the one or more infusion pumps; and (b) inspect urine measurement output data from the kidney urine output monitor for one or more urine flow parameter changes correlated to the detected or induced infusion pattern.

2. The medical device according to claim 1, wherein said control logic is configured to induce modulation onto an infusion pattern already generated by one or more infusion pumps.

3. The medical device according to claim 1, wherein a modulation induced on an infusion pattern may be selected from the group consisting of:
(a) periodic modulations, wherein the infusion rate follows a periodic pattern selected from the group consisting of sinusoidal, square, and triangular waveforms, varying in frequency and amplitude;
(b) step changes, wherein sudden increases or decreases in infusion rates are applied for a predetermined duration before returning to a baseline rate;
(c) ramp modulations, wherein the infusion rate increases or decreases gradually over a set period, followed by a return to the initial rate, and wherein the ramp can be linear or nonlinear;
(d) pulse modulations, comprising short bursts of increased or decreased infusion rates interspersed with periods of baseline infusion, varying in duration and intensity;
(e) random or stochastic modulations, wherein the infusion rates are varied in a random or pseudo-random manner to introduce unpredictability into the infusion pattern;
(f) combinations thereof, wherein any combination of periodic, step, ramp, pulse, and stochastic modulations are employed to create complex infusion patterns; and
(g) patient-specific modulations, tailored to the individual patient's current condition and historical response data, taking into account the patient's specific physiological characteristics and past responses to fluid administration.

4. The device according to claim 1, wherein the one or more urine flow parameter changes monitored for include: (a) Time to Response; (b) Rate of Urine Output;
(c)Volume of Urine Output; (d) Slope of Urine Rate Change; (e) Amplitude of Urine Rate Change; (f) Curvature or Second Derivative of the Urine Rate Change; (g) Differences in Response to Positive versus Negative Infusion Rate Changes; (h) Urine Specific Gravity or Osmolality Changes; (i) Variability in Urine Output.
